(19) United States
(12) Patent Application Publication
(10) Pub. No.: US 2023/0250432 A1
(43) Pub. Date: Aug. 10, 2023

(54) siRNA-Copolymer Compositions And Methods Of Use For Treatment Of Liver Cancer

(71) Applicant: SIRNAOMICS, INC., Gaithersburg, MD (US)

(72) Inventors: Michael MOLYNEAUX, Gaithersburg, MD (US); Patrick LU, Gaithersburg, MD (US)

(21) Appl. No.: 17/867,260

(22) Filed: Jul. 18, 2022

Related U.S. Application Data
(60) Provisional application No. 63/222,952, filed on Jul. 16, 2021.

(51) Int. Cl. C12N 15/113; A61P 35/00; C07K 16/28; C07K 16/18

(52) U.S. Cl. CPC C12N 15/1137; A61P 35/00; C07K 16/18; C07K 16/2818; C12N 15/1136; C12N 2310/11

(57) ABSTRACT

Compositions and methods for treating hepatocellular carcinoma (HCC) using siRNA molecules are provided. The compositions advantageously are administered in nanoparticle form, where the nanoparticles also contain a histidine-lysine copolymer (“HKP”). In specific embodiments, the composition contains an siRNA molecule that targets TGF-β1, an siRNA molecule that targets Cox-2, and an HKP copolymer.

### CROSS-REFERENCE TO RELATED PATENT APPLICATIONS

This application claims the benefit of and priority to U.S. Provisional Pat. Application No. 63/222,952, filed Jul. 16, 2021, which is incorporated herein by reference in its entirety.

### SEQUENCE LISTING

The instant application contains a Sequence Listing submitted electronically in ST.26 (XML) format and hereby incorporated by reference in its entirety. The XML file, created on Sep. 6, 2022, is named 0048c_ST_26 and is 7 kilobytes in size.

### FIELD

Compositions containing siRNA molecules combined with a copolymer carrier are provided, together with methods for targeting and modulating the expression of genes in the treatment of liver cancer, such as hepatocellular carcinoma.

### BACKGROUND

Hepatocellular carcinoma (HCC) is an inflammation-induced and chemotherapy-resistant cancer. Dysregulated signaling in the transforming growth factor beta (TGFβ) pathway plays a role in inflammation, fibrogenesis, and immunomodulation in the HCC microenvironment. TGFβ and Cox2 upregulation each play a negative role in inducing inflammation and also in converting active T-cells to inactive T-reg cells — reducing their ability to attack tumor cells.

### SUMMARY

What is provided is a method for treating liver cancer in a subject by to the subject a therapeutically effective amount of a pharmaceutical composition containing an siRNA that targets TGF-β1, an siRNA that targets COX-2, and a pharmaceutically effective carrier. The pharmaceutically acceptable carrier may be an HKP, such HK34b or HK34b(+H).

The siRNA that targets TGF-β1 may be a duplex of SEQ ID NO:1 and SEQ ID NO:2, and the siRNA that targets COX-2 may be a duplex of SEQ. ID NO: 3 and SEQ. ID NO: 4.

The liver cancer may be, for example, hepatocellular carcinoma (HCC), or it may be a tumor that has metastasized from another tumor site in the body outside the liver.

The composition may be administered intratumorally, or may be administered intravenously or through an intraperitoneal route. The composition may also be administered with an immune checkpoint inhibitor, which may be an antibody, an agent that binds to or otherwise inhibits a checkpoint protein selected from the group consisting of PD-1, PDL1, Lag3, Tim3, and CTLA-4/B7; a CTLA-4 inhibitor; a lymphocyte activation gene-3 (LAG-3) inhibitor; and an immune checkpoint inhibitor that targets (i) T cell immunoglobulin and mucin-domain containing-3 (TIM-3); (ii) T cell immunoglobulin and ITIM domain (TIGIT), (iii) V-domain Ig suppressor of T cell activation (VISTA); (iv) B7 homolog 3 protein (B7-H3); or (v) B and T cell lymphocyte attenuator (BTLA)). The immune checkpoint inhibitor may be, for example,: a PD-1 inhibitor selected from the group consisting of Pembrolizumab (Keytruda), Nivolumab (Opdivo), and Cemiplimab (Libtayo); a PD-1 inhibitor selected from the group consisting of Atezolizumab (Tecentriq), Avelumab (Bavencio), and Durvalumab (Imfinzi); Ipilimumab (Yervoy); or BMS-986016.

### BRIEF DESCRIPTION OF THE DRAWINGS

FIGS. 1(a)-1(c) show the effect of STP705 treatment on hepatocellular carcinoma (HCC) in mice.

FIG. 2 shows TGFβ levels in STP705 treatment versus control (vehicle only) groups for Huh7 xenograft tumor samples. The final siRNA treatment injection took place on day 24. Xenograft tumor samples were collected at the end of the experiment (day 35) and were subject to two-step real-time PCR.

FIG. 3 shows assessment of the intrahepatic multiple dose toxicity of STP705 in C57BL/6 mice using a dosing regimen for testing leakage after local injection.

FIG. 4 shows no effect on hematology and serum parameters at 24 hours and 14 days following STP705 administration.

FIG. 5 shows no effect on liver enzyme levels at 24 hours and 14 days following STP705 administration.

FIG. 6 shows that hematologic and liver function parameters in STP705-treated groups remain within normal ranges (see O′Connell, KE, et. al. “Practical Murine Hematopathology: A Comparative Review and Implications for Research,” Comp. Med. 65(2), 2015).

### DETAILED DESCRIPTION

Compositions and methods for treating hepatocellular carcinoma (HCC) using siRNA molecules are provided. The compositions advantageously are administered in nanoparticle form, where the nanoparticles also contain a histidine-lysine copolymer (“HKP”). In specific embodiments, the composition contains an siRNA molecule that targets TGF-β1, an siRNA molecule that targets Cox-2, and an HKP copolymer.

siRNA molecules that target TGF-β1 and Cox-2 are well known in the art and are described in, for example, U.S. Pat. No. 9,642,873. Advantageously, the siRNAs have the sequences shown below:

### TGF-β1

Sense strand: 5′ CCC AAG GGC UAC CAU GCC AAC UUC U 3′ (SEQ ID NO:1)

Antisense strand: 5′ AGA AGU UGG CAU GGU AGC CCU UGG G 3 (SEQ ID NO:2)

### Cox-2

Sense strand: 5′ GGU CUG GUG CCU GGU CUG AUG AUG U 3′ (SEQ ID NO:3)

Antisense strand 5′ ACA UCA UCA GAC CAG GCA CCA GAC C 3′ (SEQ ID NO:4)

Advantageously the HKP copolymer is H3K4b, which is a branched peptide with a backbone of three L-lysine residues, where the N-terminus and the three lysine ε-amino groups are linked to a histidine-lysine peptide chain with the structure KH₃KH₃KH₃KH₃ (SEQ ID NO:5). The C-terminus of the peptide is amidated. The structure of H3K4b is shown below:

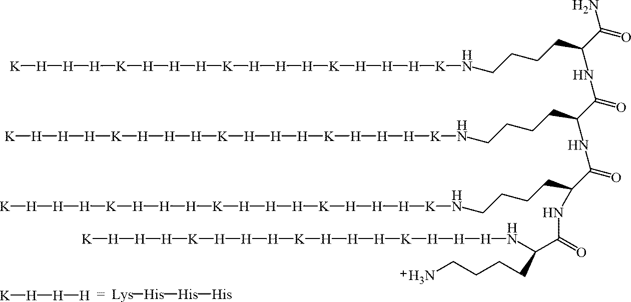

The HKP polymer also may be H3K4b(+H) which has a similar structure to that shown above except that the histidine-lysine peptide chain contains an additional histidine residue: KH₃KH₄[KH₃]₂K. Compositions using H3K4b(+H) may be used for systemic administration, for example.

Use of a nanoparticle containing two siRNAs targeting TGFβ1 and Cox-2 allows simultaneous co-delivery into the same cell, silencing both (TGFβ and Cox2) targets, and resulting in antitumoral activity in HCC. Intratumoral administration of the two siRNAs in a nanoparticle delivery system also significantly impacts the development of HCC tumors. Direct local injection into the tumor has therapeutic value in both HCC and cholangiocarcinoma (CCA), and other cancers as mentioned infra. Systemic administration also allows uptake of the nanoparticles directly into tumors, including in those that have metastasized to the liver tissue. Such tumors that have metastasized to the liver may include cancers originating in the colon, esophageal, stomach, pancreatic, lung, kidney, breast and skin.

In some embodiments the compositions may be administered through intratumoral injection; in other embodiments the compositions are administered systemically. In certain embodiments administration is through systemic intravenous injection or infusion. The compositions also may be administered together with an effective amount of an immune checkpoint inhibitor for additive effect. Suitable immune checkpoint inhibitors are known in the art, and typically contain an antibody or other agent that binds to or otherwise inhibits a checkpoint protein selected from the group consisting of PD-1, PDL1, Lag3, Tim3, and CTLA-4/B7. The checkpoint inhibitor may be, for example: a PD-1 inhibitor, such as Pembrolizumab (Keytruda), Nivolumab (Opdivo), or Cemiplimab (Libtayo); a PD-L1 inhibitor such as Atezolizumab (Tecentriq), Avelumab (Bavencio), and Durvalumab (Imfinzi); a CTLA-4 inhibitor such as Ipilimumab (Yervoy); a lymphocyte activation gene-3 (LAG-3) inhibitor such as BMS-986016; or may be an immune checkpoint inhibitor that targets T cell immunoglobulin and mucin-domain containing-3 (TIM-3), T cell immunoglobulin and ITIM domain (TIGIT), V-domain Ig suppressor of T cell activation (VISTA), B7 homolog 3 protein (B7-H3) or B and T cell lymphocyte attenuator (BTLA)).

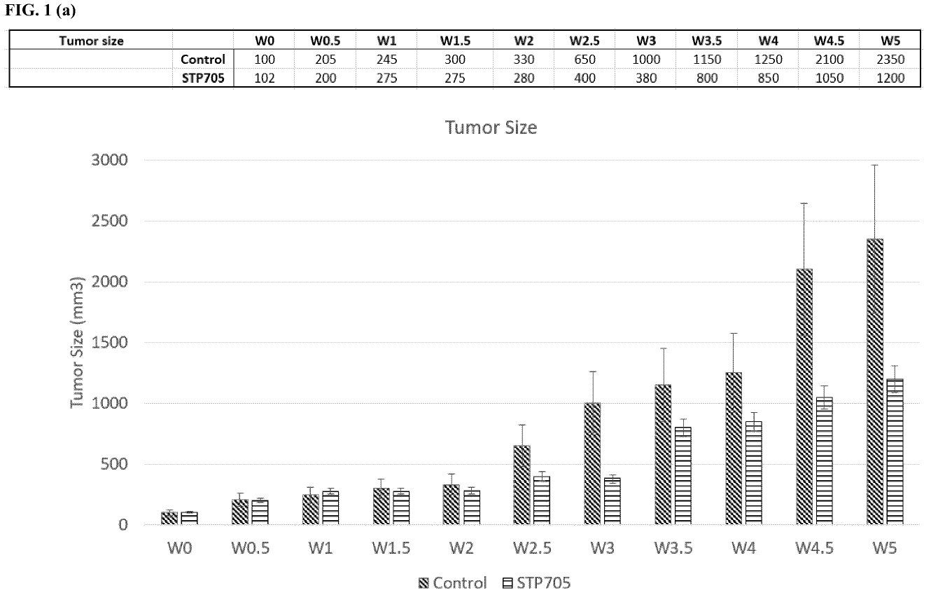

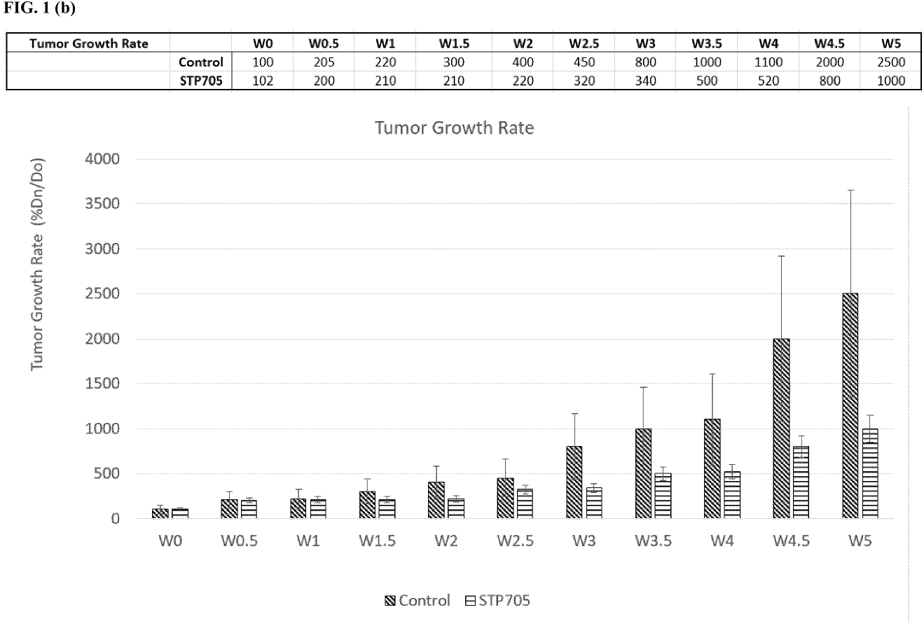

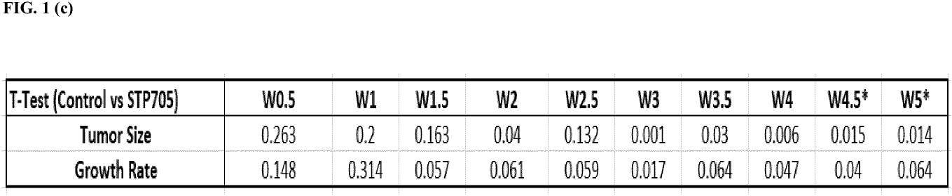

As described in more detail in Example 1 below, the composition was administered directly into HCC tumors in xenograft studies in mice, causing a significant reduction in the size of the tumors and the growth rate of the tumors (FIGS. 1).

The sequences of the sense strand of the TGFβ1 and Cox2 siRNAs are shown below along with the sequences of the same genes in humans, mice, monkeys and pigs. The siRNA sequences have identity to the genes in humans, mice and monkeys. Cox2 siRNA also has identity with the gene in pigs. TGFβ1 siRNA has identity with the sequence in pig barring a single nucleotide (C—U).

The sequences described below are the sense strands of blunt-ended double stranded RNA molecules. The skilled artisan will appreciate that the siRNA molecules contain the sense strand as shown as part of a duplex with its complementary sequence. Reference herein to the siRNA molecule of SEQ ID NO:X will be understood to refer to the duplex formed by the sense strand (SEQ ID NO:X) and the corresponding antisense strand.

As used herein, silencing a gene means reducing the concentration of the mRNA transcript of that gene, which reduces the concentration of the protein product of that gene by at least 10%, at least 20%, at least 30%, at least 40%, at least 50%, at least 70%, at least 80% or at least 90% or more.

| | | |
|---|---|---|
| | TGF-β1 | COX-2 |
| siRNA | 5′-CCCAAGGGCUACCAUGCCAACUUCU-3′ | 5′-GGUCUGGUGCCUGGUCUGAUGAUGU-3′ |
| Human | 5′-CCCAAGGGCUACCAUGCCAACUUCU-3′ | 5′-GGUCUGGUGCCUGGUCUGAUGAUGU-3′ |
| Mouse | 5′-CCCAAGGGCUACCAUGCCAACUUCU-3′ | 5′-GGUCUGGUGCCUGGUCUGAUGAUGU-3′ |
| Monkey | 5′-CCCAAGGGCUACCAUGCCAACUUCU-3′ | 5′-GGUCUGGUGCCUGGUCUGAUGAUGU-3′ |
| Pig | 5′-CCCAAGGGCUACCAUGCCAAuUUCU-3 | 5′-GGUCUGGUGCCUGGUCUGAUGAUGU-3′ |

In each of these siRNA molecules, one or more of the nucleotides in either the sense or the antisense strand can be modified. Modified nucleotides can improve stability and decrease immune stimulation by the siRNAs. The modified nucleotide may be, for example, a 2′—O—methyl, 2′-methoxyethoxy, 2′-fluoro, 2′-allyl, 2′—O—[2-(methylamino)-2-oxoethyl], 4′-thio, 4′—CH2—O—2′-bridge, 4′—(CH2)2—O—2′-bridge, 2′-LNA, 2′-amino or 2′—O—(N—methylcarbamate) ribonucleotide. One, two, three, four, five, six, seven, eight, nine, ten, eleven, twelve, thirteen, fourteen, fifteen, sixteen, seventeen, eighteen, nineteen, twenty, twenty-one, twenty-two, twentythree, twenty-four or twenty-five of the nucleotides may be modified. When multiple modifications are made in an siRNA strand, the modifications may be the same or different in the same strand.

In addition, one or more of the phosphodiester linkages between the ribonucleotides may be modified to improve resistance to nuclease digestion. Suitable modifications include the use of phosphorothioate and/or phosphorodithioate modified linkages.

### Formation of Nanoparticles Containing siRNAs Targeting TGFβ1 and Cox2

The siRNA molecules containing the described above are formulated into nanoparticles for administration to a subject. Various methods of nanoparticle formation are well known in the art. See, for example, Babu et al., IEEE Trans Nanobioscience, 15: 849-863, 2016.

Advantageously, the nanoparticles are formed using one or more histidine/lysine (HKP) copolymers. Suitable HKP copolymers are described in WO/2001/047496, WO/2003/090719, and WO/2006/060182, the contents of each of which are incorporated herein in their entireties. HKP copolymers form a nanoparticle containing an siRNA molecule, typically 100-400 nm in diameter. HKP and HKP(+H) both have a lysine backbone (three lysine residues) where the lysine side chain ε-amino groups and the N-terminus are coupled to [KH₃]₄K (for HKP) or KH₃KH₄[KH₃]₂K (for HKP(+H). The branched HKP carriers can be synthesized by methods that are well-known in the art including, for example, solid-phase peptide synthesis.

Methods of forming nanoparticles are well known in the art. Babu et al., supra. Advantageously, nanoparticles may be formed using a microfluidic mixer system, in which an siRNA targeting TGFβ1 and an siRNA targeting Cox2 are mixed with one or more HKP polymers at a fixed flow rate. The flow rate can be varied to vary the size of the nanoparticles produced.

### Determination of Efficacy of the siRNA Molecules

Depending on the particular target TGFβ1 and Cox2 RNA sequences and the dose of the nanoparticle composition delivered, partial or complete loss of function for the TGFβ1 and Cox2 RNAs may be observed. A reduction or loss of RNA levels or expression (either TGFβ1 and Cox2 RNA expression or encoded polypeptide expression) in at least 50%, 60%, 70%, 80%, 90%, 95% or 99% or more of targeted cells is exemplary. Inhibition of TGFβ1 and Cox2RNA levels or expression refers to the absence (or observable decrease) in the level of TGFβ1 and Cox2 RNA or TGFβ1 and Cox2 RNA-encoded protein. Specificity refers to the ability to inhibit the TGFβ1 and Cox2 RNA without manifest effects on other genes of the cell. The consequences of inhibition can be confirmed by examination of the outward properties of the cell or organism or by biochemical techniques such as RNA solution hybridization, nuclease protection, Northern hybridization, reverse transcription, gene expression monitoring with a microarray, antibody binding, enzyme linked immunosorbent assay (ELISA), Western blotting, radioimmunoassay (RIA), other immunoassays, and fluorescence activated cell analysis (FACS). Inhibition of target TGFβ1 and Cox2 RNA sequence(s) by the dsRNA agents of the disclosed embodiments also can be measured based upon the effect of administration of such dsRNA agents upon development/progression of a TGFβ1 and Cox2-associated disease or disorder, e.g., tumor formation, growth, metastasis, etc., either in vivo or in vitro. Treatment and/or reductions in tumor or cancer cell levels can include halting or reduction of growth of tumor or cancer cell levels or reductions of, e.g., 10%, 20%, 30%, 40%, 50%, 60%, 70%, 80%, 90%, 95% or 99% or more, and can also be measured in logarithmic terms, e.g., 10-fold, 100-fold, 1000-fold, 10⁵-fold, 10⁶-fold, or 10⁷-fold reduction in cancer cell levels could be achieved via administration of the nanoparticle composition to cells, a tissue, or a subject.

### Pharmaceutical Compositions and Methods of Administration

The nanoparticle compositions may be further formulated as a pharmaceutical composition using methods that are well known in the art. The composition may be formulated to be compatible with its intended route of administration. Examples of routes of administration to the subject, which is a mammal and may be a human, include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerin, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL® (BASF, Parsippany, N.J.) or phosphate buffered saline (PBS). In all cases, the composition must be sterile and should be fluid to the extent that easy syringeability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, trehalose, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in a selected solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

The compositions may also be prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Such formulations can be prepared using standard techniques. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

### Mode of Administration of STP705 for Intratumoral Delivery

STP705 may be administered using intratumoral injection in the interventional radiology suite under strictly controlled conditions. In an exemplary embodiment, interventional guided procedures are performed using either CT or Ultrasound (US) guidance for intratumoral (IT) injections. Patients may undergo a pre-procedure US, CT, MRI or PET/CT scan to evaluate the best approach for STP705 administration to avoid the needle impacting major structures or leading to the needle crossing multiple anatomic compartments (i.e., needle pathway that crosses the pleura or hollow organ), and avoiding proximity to large vessels, large areas of necrosis or high risk for a local toxicity event such as bleeding.

The effect of treatment on tumor size may be evaluated using similar imaging techniques (US, CT, MRI or PET/CT). Additionally, biomarkers predictive of the growth or stage of the tumor progression can be monitored to determine the impact of treatment on outcome. Biomarkers such as AFP have been used to determine early stage HCC [2]. Furthermore, the TGFβ signature may be used as a potential biomarker for identifying the ‘exhausted’ immune signature in HCC [1] and, therefore, silencing the TGFβ within the tumor or its immediate vicinity (the tumor microenvironment or TME) may alter this signature and indicate therapeutic effect. Additional biomarkers may also be used to study therapeutic efficacy or to select patients for appropriate treatment [3].

In other embodiments, suitably formulated pharmaceutical compositions as described herein may be administered by means known in the art such as by parenteral routes, including intravenous, intramuscular, intraperitoneal, subcutaneous, transdermal, airway (aerosol), rectal, vaginal and topical (including buccal and sublingual) administration. Advantageously, the pharmaceutical compositions are administered by intravenous or intraparenteral infusion or injection.

### Determination of Dosage and Toxicity

Toxicity and therapeutic efficacy of the compositions may be determined by standard pharmaceutical procedures in cell cultures or experimental animals, e.g., by determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀.

Data from cell culture assays and animal studies can be used in formulating a range of dosage for use in humans. The dosage of the compositions advantageously is within a range of circulating concentrations that include the ED50 with little or no toxicity. The dosage may vary within this range depending upon the dosage form employed and the route of administration utilized. For the compositions described herein, a therapeutically effective dose can be estimated initially from cell culture assays. A dose may be formulated in animal models to achieve a circulating plasma concentration range that includes the IC50 (i.e., the concentration of the composition which achieves a half-maximal inhibition of symptoms) as determined in cell culture. Such information can be used to more accurately determine useful doses in humans. Levels in plasma may be measured, for example, by high performance liquid chromatography (HPLC).

As used herein, a pharmacologically or therapeutically effective amount refers to that amount of an siRNA composition effective to produce the intended pharmacological, therapeutic or preventive result. The phrases “pharmacologically effective amount” and “therapeutically effective amount” or “effective amount” refer to that amount of the composition effective to produce the intended pharmacological, therapeutic or preventive result. For example, if a given clinical treatment is considered effective when there is at least a 30% reduction in a measurable parameter associated with a disease or disorder, a therapeutically effective amount of a drug for the treatment of that disease or disorder is the amount necessary to effect at least a 30% reduction in that parameter.

A therapeutically effective amount of a composition as described herein can be in the range of approximately 1 picograms (pg) to 1000 milligrams (mg). For example, 10, 30, 100, or 1000 pg, or 10, 30, 100, or 1000 nanograms (ng), or 10, 30, 100, or 1000 micrograms (µg), or 10, 30, 100, or 1000 mg, or 1-5 g of the compositions can be administered. In general, a suitable dosage unit of the compositions described herein will be in the range of 0.001 to 0.25 mg per kilogram (kg) body weight of the recipient per day, or in the range of 0.01 to 20 µg per kg body weight per day, or in the range of 0.001 to 5 µg per kg of body weight per day, or in the range of 1 to 500 ng per kg of body weight per day, or in the range of 0.01 to 10 µg per kg body weight per day, or in the range of 0.10 to 5 µg per kg body weight per day, or in the range of 0.1 to 2.5 µg per kg body weight per day. The pharmaceutical composition can be administered once daily, or may be dosed in dosage units containing two, three, four, five, six or more sub-doses administered at appropriate intervals throughout the day. In that case, the dsRNA contained in each sub-dose must be correspondingly smaller in order to achieve the total daily dosage unit. The dosage unit can also be compounded for a single dose over several days, e.g., using a conventional sustained release formulation which provides sustained and consistent release of the dsRNA over a several day period. Sustained release formulations are well known in the art. In this embodiment, the dosage unit contains a corresponding multiple of the daily dose. Regardless of the formulation, the pharmaceutical composition must contain dsRNA in a quantity sufficient to inhibit expression of the target gene in the animal or human being treated. The composition can be compounded in such a way that the sum of the multiple units of dsRNA together contain a sufficient dose.

The compositions may be administered once, one or more times per day to one or more times per week; including once every other day. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of a composition as described herein may include a single treatment or, advantageously, can include a series of treatments.

### Treatment for HCC and Other Proliferative Diseases

The compositions described herein may be used to treat proliferative diseases, such as cancer, and particularly liver cancer, characterized by expression, and particularly altered expression, of TGFβ1 and Cox2. Exemplary cancers include liver cancer, such as hepatocellular carcinoma or HCC, and tumor tissue in the liver that has metastasized from other tumors, such as lung cancer (e.g., NSCLC), colorectal cancer, prostate cancer, pancreatic cancer, ovarian cancer, cervical cancer, brain cancer (e.g., glioblastoma), renal cancer (e.g., papillary renal carcinoma), stomach cancer, esophageal cancer, medulloblastoma, thyroid carcinoma, rhabdomyosarcoma, osteosarcoma, squamous cell carcinoma (e.g., oral squamous cell carcinoma), melanoma, breast cancer, and hematopoietic disorders (e.g., leukemias and lymphomas, and other immune cell-related disorders.

The compositions may be administered as described above and, advantageously may be delivered systemically or intratumorally. The compositions may be administered as a monotherapy, i.e. in the absence of another treatment, or may be administered as part of a combination regimen that includes one or more additional medications. Advantageously, when used as part of a combination regimen that includes an effective amount of at least one additional chemotherapy drug. The chemotherapy drug may be, for example, a platinum-containing drug, such as cisplatin, oxaloplatin, or carboplatin.

All ranges and ratios discussed here can and necessarily do describe all subranges and subratios therein for all purposes, and all such subranges and subratios also form part and parcel of the disclosed embodiments. Any listed range or ratio can be easily recognized as sufficiently describing and enabling the same range or ratio being broken down into at least equal halves, thirds, quarters, fifths, tenths, etc. As a non-limiting example, each range or ratio discussed herein can be readily broken down into a lower third, middle third and upper third, etc.

The disclosed embodiments will be better understood by reference to the following examples which are intended for purposes of illustration and are not intended to be interpreted in any way to limit the scope of the appended claims.

The word “exemplary” is used herein to mean “serving as an example, instance, or illustration.” Any embodiment described herein as “exemplary” is not necessarily to be construed as preferred or advantageous over other embodiments.

Recitation in the claims of the term “first” with respect to a feature or element does not necessarily imply the existence of a second or additional such feature or element. Elements recited in means-plus-function format are intended to be construed in accordance with 35 U.S.C. § 112 ¶ 6. It will be apparently to those having skill in the art that changes may be made to the details of the above-described embodiments without departing from the underlying principles of the disclosed embodiments.

While specific embodiments and application of the disclosed embodiments have been illustrated and described, it is to be understood that the disclosed embodiments are not limited to the precise configuration and components disclosed herein. Various modifications, changes, and variations, which will be apparent to those skilled in the art may be made in the arrangement, operation, and details of the methods and systems of the embodiments disclosed herein, including those of the appended claims. Finally, various features of the disclosed embodiments herein may be combined to provide additional configurations which fall within the scope of the disclosed embodiments. The following examples are intended to illustrate the kinetic measures and the efficacy of inhibitory compounds tested, including those in the disclosed embodiments.

### Example 1

STP705 comprises siRNA molecules targeting TGFβ (a duplex of the siRNAs of SEQ ID NOs 1 and 2) and Cox2 (a duplex of the siRNAs of SEQ ID NOs: 3 and 4) formulated together with HKP copolymer in nanoparticle form. The siRNAs were mixed at 0.5 mg/ml with HKP(+H) using a PNI microfluidic mixer system (Precision Nanosystems, Inc., Vancouver, CA). Total Flow Rate (TFR) was varied and the effect of this flow rate on particle size was evaluated by measuring resulting particle size using a Malvern Nanosizer system (Malvern Panalytical Inc., Westborough, MA). The polydispersity index (PDI) is an indication of the amount of variation of the nanoparticles around the average size.

HCC Cells (Huh7 with a TERT promoter mutation -124 G->A) with stable Luciferase expression were harvested by Accutase® and density was adjusted to 25 million cells/mL in PBS. 0.2 mL cell solution was injected subcutaneously to both flanks of NOD-SCID mice. Tumor size was measured twice a week (Dmax*Dmin²/2). When the tumor size reached >100 mm³, dosing of animals was started by direct intratumoral injection of 50uL siRNA complex (STP705) into each tumor. If a mouse had 2 tumors, the same formula was injected into both sites. The siRNA formulation was a mixture of synthetic siRNA (TGFβ1) and siRNA (COX-2) (8 µg each) supplemented with 64ug HKP and dissolved in 50uL ddH2O. Tumors were treated twice a week through the 8^{th} dosing and tumor size was measured during treatment until week 5. Animals were sacrificed and tumors collected for weight and for H/E stain and Q-PCR analyses. The upper figure shows the effect of administration of STP705 on tumor size (FIG. 1 (a)) while the lower figure shows the effect of STP705 on growth rate (FIG. 1 (b)) of the tumor. The lower table (FIG. 1 (c)) shows the T-test values obtained comparing between test and control treated samples in tumor size and growth rate. In both cases a statistically significant difference was noted and STP705 inhibits Huh7 xenograft growth from week 1.5 to week5 (p<0.05).

Administration of STP705 to mice with HCC tumor xenograft implants resulted in a significant reduction in the size of the tumor over time as well as the growth rate of the tumor with time (FIGS. 1).

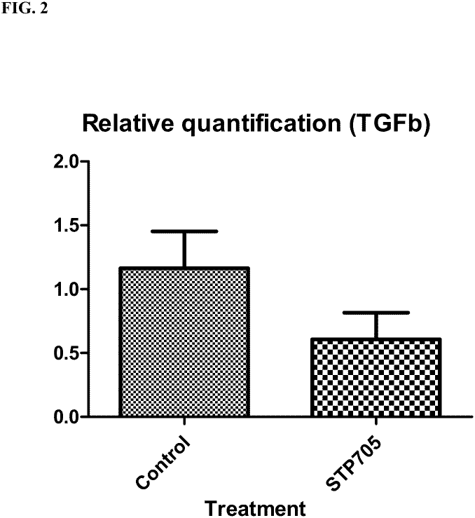

The observed reduction in the level of TGFβ1 expression in tissue treated with STP705 shows that the siRNA is delivered to the tissue and the cells within the tissue and can silence the expression of the protein (FIG. 2).

### Example 2

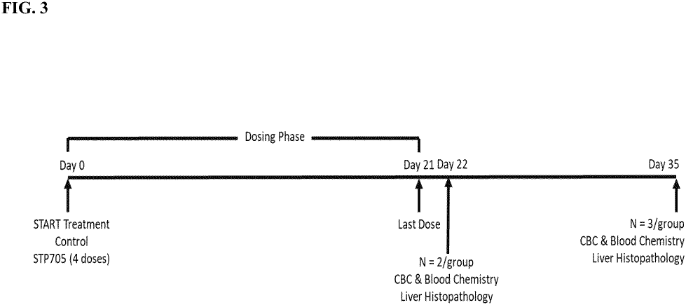

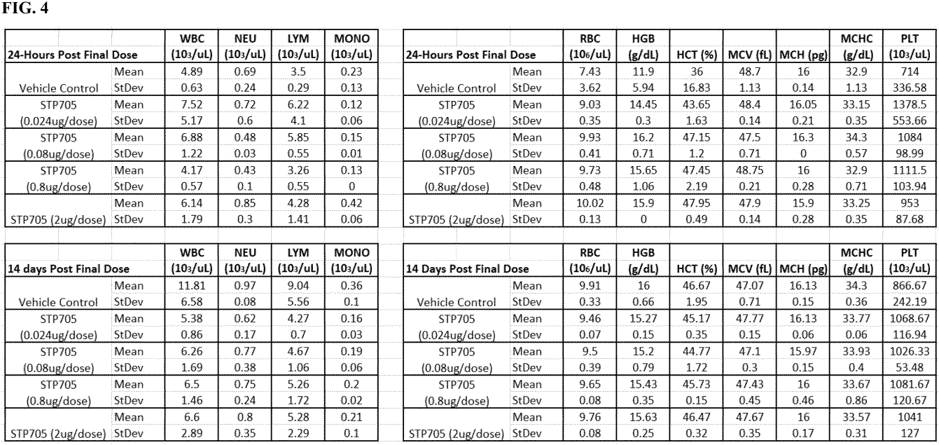

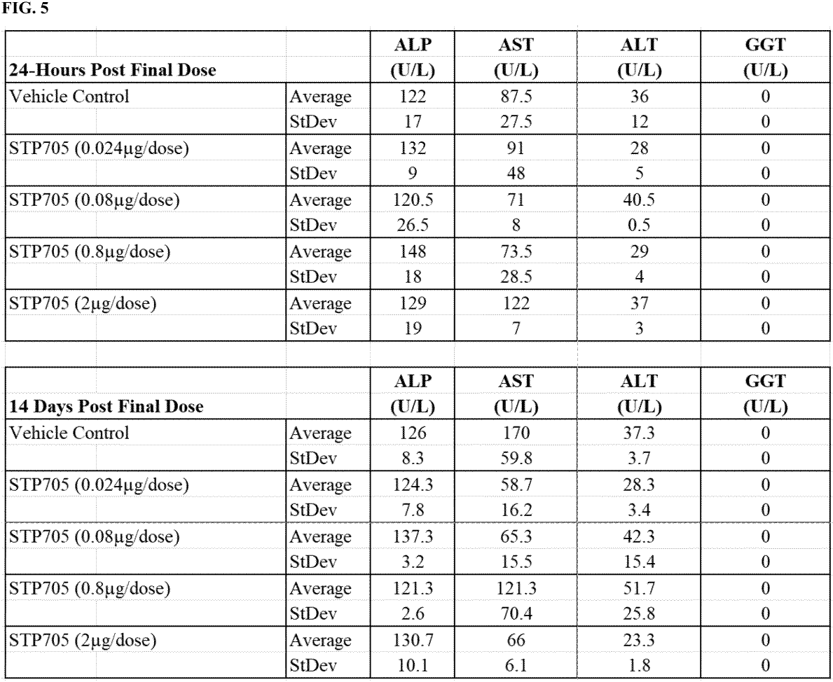

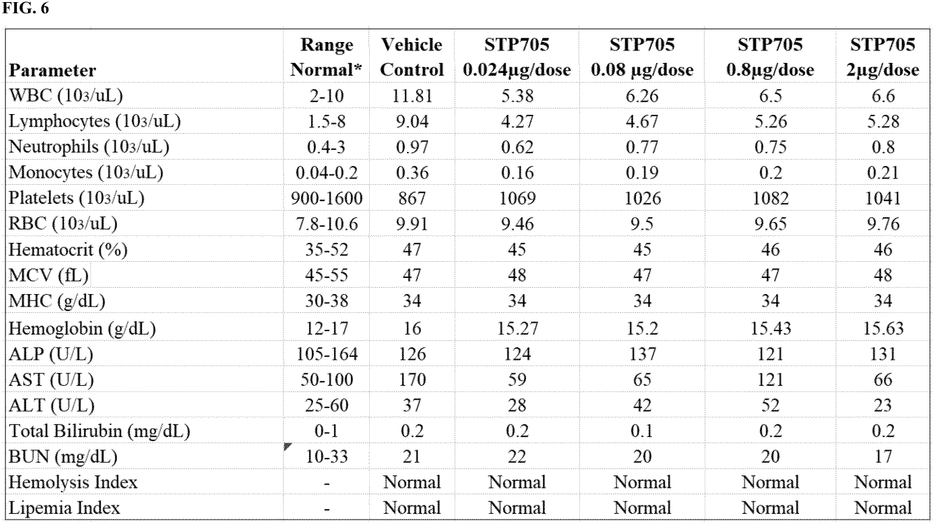

To ensure that the STP705 did not produce an effect through non-specific means, we examined the impact of STP705 administration on normal hepatic tissue when administered intrahepatically via percutaneous injection (FIG. 3). This mode of administration had no effect on hematology and serum analyses (FIG. 4) or on liver enzyme levels (FIG. 5). STP705 treatment did not result in abnormal liver enzyme levels (FIG. 6).

Further, no significant differences were observed between each of the STP705 treatment groups (2 through 5) and the control group (1) in the gallbladder’s mean mucosal neutrophilic infiltrate score at 24-hours and 14-days following administration of the final dose.

Acute coagulative hepatocellular necrosis was observed in both the vehicle control group of mice and the STP705-administered mice with no consistent differences in incidence or severity, indicating an association with the injection type (intrahepatic) rather than the test article being injected.

Subacute to chronic hepatocellular loss was observed in all groups, including vehicle control with no consistent differences in incidence or severity. This finding is considered to be associated with intrahepatic injection and not administration of the test article. Lesion severity was minimal to mild, suggesting the long-term effects on the hepatic parenchyma following intrahepatic injection are low.

The pathological assessment of stained liver tissue sections by a board-certified veterinary pathologist indicated there were no significant differences observed between the control and STP705 treated groups for pathologic parameters (including hepatocellular necrosis, acute/chronic lesions, inflammation, granuloma). The clinical pathology parameters (including hematology and serum chemistry) indicated that there were no differences between the control and STP705 treated animals at any of the time points. The complete blood count (CBC) and liver function test results for the control and STP705-treated animals were within the normal range (as reported in the literature).

Overall, in a normal C57BL/6 murine model, the study indicated that intrahepatic delivery of STP705 dose up to 2 µg (that is comparable to a 2000 µg human dose) is well tolerated, with no apparent signs of liver toxicity, which supports the utility of this treatment option for treating HCC and other liver conditions.

In addition to the ability to impact HCC in the liver the therapeutic use of STP705 may have utility in treating tumors from other organs that have metastasized to the liver, for example, in colon, esophageal, stomach, pancreatic, lung, kidney, breast and skin cancers.

### REFERENCES

Immunomodulatory TGF-b Signaling in Hepatocellular Carcinoma. Jian Chen et al., Trends in Molecular Medicine, November 2019, Vol. 25, No. 11. Doi: Https://doi.org/10.1016/j.molmed.2019.06.007

Biomarkers for the Early Detection of Hepatocellular Carcinoma (2020), Neehar D. Parikh et al., Cancer Epidemiol Biomarkers Prev 2020;29:2495-503. doi: 10.1158/1055-9965.EPI-20-0005

Biomarkers in Hepatocellular Carcinoma: Diagnosis, Prognosis and Treatment Response Assessment (2020). Federico Piñero et al., Cells. Jun; 9(6): 1370. doi: 10.3390/cells9061370

What is claimed is:

1. A method for treating liver cancer in a subject comprising administering to said subject a therapeutically effective amount of a pharmaceutical composition comprising an siRNA that targets TGF-β1, an siRNA that targets COX-2, and a pharmaceutically effective carrier.

2. The method according to claim 1 wherein said pharmaceutically acceptable carrier comprises an HKP.

3. The method according to claim 2, wherein said HKP is HK34b or HK34b(+H).

4. The method according to any preceding claim wherein said siRNA that targets TGF-β1 comprises SEQ ID NO:1 and SEQ ID NO:2.

5. The method according to claim 1, wherein siRNA that targets COX-2 comprises SEQ. ID NO: 3 and SEQ. ID NO: 4.

6. The method according to claim 1, wherein the liver cancer is hepatocellular carcinoma (HCC).

7. The method according to claim 1, wherein the liver cancer is a tumor that has metastasized from another tumor site in the body outside the liver.

8. The method according to claim 1, wherein said composition is administered intratumorally.

9. The method according to claim 1, wherein said composition is administered intravenously or through an intraperitoneal route.

10. The method according to claim 1 wherein said composition is administered with an immune checkpoint inhibitor.

11. The method of claim 10 wherein said immune checkpoint inhibitor is selected from the group consisting of an antibody, an agent that binds to or otherwise inhibits a checkpoint protein selected from the group consisting of PD-1, PDL1, Lag3, Tim3, and CTLA-4/B7; a CTLA-4 inhibitor; a lymphocyte activation gene-3 (LAG-3) inhibitor; and an immune checkpoint inhibitor that targets (i) T cell immunoglobulin and mucin-domain containing-3 (TIM-3); (ii) T cell immunoglobulin and ITIM domain (TIGIT), (iii) V-domain Ig suppressor of T cell activation (VISTA); (iv) B7 homolog 3 protein (B7-H3); or (v) B and T cell lymphocyte attenuator (BTLA)).

12. The method according to claim 11, wherein the immune checkpoint inhibitor is a PD-1 inhibitor selected from the group consisting of Pembrolizumab (Keytruda), Nivolumab (Opdivo), and Cemiplimab (Libtayo).

13. The method according to claim 11, wherein the immune checkpoint inhibitor is a PD-1 inhibitor selected from the group consisting of Atezolizumab (Tecentriq), Avelumab (Bavencio), and Durvalumab (Imfinzi).

14. The method according to claim 11, wherein the immune checkpoint inhibitor is Ipilimumab (Yervoy).

15. The method according to claim 11, wherein the immune checkpoint inhibitor is BMS-986016.


SEQUENCE LISTING (5 sequences)

SEQ ID NO: 1 - DNA, 25 bp, synthetic construct
  misc_feature 1..25: note = synthetic construct 1
  source 1..25: mol_type = other DNA
cccaagggctaccatgccaacttct

SEQ ID NO: 2 - DNA, 25 bp, synthetic construct
  misc_feature 1..25: note = synthetic construct 2
  source 1..25: mol_type = other DNA
agaagttggcatggtagcccttggg

SEQ ID NO: 3 - DNA, 25 bp, synthetic construct
  misc_feature 1..25: note = synthetic construct 3
  source 1..25: mol_type = other DNA
ggtctggtgcctggtctgatgatgt

SEQ ID NO: 4 - DNA, 25 bp, synthetic construct
  misc_feature 1..25: note = synthetic construct
  source 1..25: mol_type = other DNA
acatcatcagaccaggcaccagacc

SEQ ID NO: 5 - protein, 16 aa, synthetic construct
  REGION 1..16: note = synthetic construct
KHHHKHHHKHHHKHHH